(19) United States
(12) Patent Application Publication
(10) Pub. No.: US 2019/0125201 A1
(43) Pub. Date: May 2, 2019

(54) METHOD AND APPARATUS FOR MEASURING BLOOD PRESSURE USING OPTICAL SENSOR

(71) Applicant: ONESOFTDIGM CO., LTD, Pohang-si (KR)

(72) Inventors: Dae Ho LEE, Pohang-si (KR); Sung Min PARK, Pohang-si (KR)

(21) Appl. No.: 16/172,555

(22) Filed: Oct. 26, 2018

(30) Foreign Application Priority Data
Oct. 31, 2017 (KR) 10-2017-0144149

(51) Int. Cl. A61B 5/0225; A61B 5/021; A61B 5/0456; A61B 5/00

(52) U.S. Cl. CPC A61B 5/02255; A61B 5/02125; A61B 5/02225; A61B 5/7239; A61B 5/0456

(57) ABSTRACT

A method of measuring blood pressure is disclosed. The method includes receiving electrocardiogram information and blood flow rate information from a first sensor module, calculating a pulse transit time on the basis of the electrocardiogram information and the blood flow rate information, calculating a mean blood pressure value of a user on the basis of the pulse transit time, receiving a reference data from a second sensor module, and calculating a diastolic blood pressure value and a systolic blood pressure value of the user on the basis of the mean blood pressure value and the reference data. Because the present invention continuously measures blood pressure for 24 hours in a non-pressing manner, there is an effect in that abnormal signs of the blood pressure may be easily checked.

### CLAIM FOR PRIORITY

This application claims priority to Korean Patent Application No. 10-2017-0144149 filed on Oct. 31, 2017 in the Korean Intellectual Property Office (KIPO), the entire contents of which are hereby incorporated by reference.

### BACKGROUND

### 1. Technical Field

Example embodiments of the present invention relate to a method and an apparatus for measuring blood pressure, and more specifically, to a method and an apparatus for measuring blood pressure using an electrocardiogram (ECG) sensor and a photoplethysmogram (PPG) sensor.

### 2. Related Art

Recently, cardiovascular diseases and complications caused by blood pressure have been widely known as major causes of death. Therefore, proper blood pressure management is the most effective way to prevent serious complications such as myocardial infarction, stroke, heart failure and kidney failure, but since blood pressure varies a great deal due to location, time, behavior context, and the like, a need for continuous measurement (24 hours a day/7 days a week) exists, and in addition, various studies to check abnormal signs of blood pressure are being carried out.

In this regard, the domestic home medical device market in Korea continues to grow at an annual mean growth of over 8% due to growing interest in health and aging population. Among sixteen home medical devices, the most commonly used device is a thermometer (45.5%), and a blood pressure gauge, a personal blood glucose meter, a cupping device, and the like are the most universally used. In addition, the blood pressure gauge, the thermometer, the personal blood glucose meter, and the like are medical devices that users want to rent.

In the overseas market, the market for blood pressure gauge will continue to grow until the year 2021, and the market is forecast to grow to about 32.5 billion. In addition, it is analyzed that the main growth factor of the blood pressure monitoring market is an increase in the elderly and obese population and an increase in importance of preventive treatment for them. In addition, as of year 2015, a share of the North American blood pressure gauge market is 42% of the total medical device market, and the North American blood pressure gauge market is expected to continue to dominate over the next few years, but the Asia-Pacific region's medical device market is also expected to rapidly grow.

### SUMMARY

Accordingly, example embodiments of the present invention are provided to substantially obviate one or more problems due to limitations and disadvantages of the related art.

In some example embodiments, a method of measuring blood pressure includes receiving electrocardiogram information and blood flow rate information from a first sensor module, calculating a pulse transit time on the basis of the electrocardiogram information and the blood flow rate information, calculating a mean blood pressure value of a user on the basis of the pulse transit time, receiving a reference data from a second sensor module, and calculating a diastolic blood pressure value and a systolic blood pressure value on the basis of the mean blood pressure value and the reference data.

In other example embodiments, a blood pressure measuring apparatus includes a receiver configured to receive electrocardiogram information and blood flow rate information from a first sensor module and receive reference data from a second sensor module, a first calculator configured to calculate a pulse transit time on the basis of the electrocardiogram information and the blood flow rate information and calculate a mean blood pressure value of a user on the basis of the pulse transit time, and a corrector configured to calculate a diastolic blood pressure value and a systolic blood pressure value of the user on the basis of the mean blood pressure value and the reference data.

### BRIEF DESCRIPTION OF DRAWINGS

Example embodiments of the present invention will become more apparent by describing in detail example embodiments of the present invention with reference to the accompanying drawings, in which:

FIG. 1 is a conceptual view illustrating a blood pressure measuring apparatus according to one embodiment of the present invention;

FIG. 2 is a block diagram illustrating the blood pressure measuring apparatus according to one embodiment of the present invention;

FIG. 3 is a view illustrating a PQRST wave of an electrocardiogram (ECG) according to one embodiment of the present invention;

FIG. 4 is a conceptual view illustrating a photoplethysmogram (PPG) sensor according to one embodiment of the present invention;

FIG. 5 is a view illustrating a pulse transit time (PTT) according to one embodiment of the present invention;

FIG. 6 is a graph for describing a vibrating blood pressure measurement method according to one embodiment of the present invention; and

FIG. 7 is a flowchart illustrating a method of measuring blood pressure according to one embodiment of the present invention.

### DESCRIPTION OF EXAMPLE EMBODIMENTS

Example embodiments of the present invention are disclosed herein. However, specific structural and functional details disclosed herein are merely representative for purposes of describing example embodiments of the present invention, however, example embodiments of the present invention may be embodied in many alternate forms and should not be construed as limited to example embodiments of the present invention set forth herein.

Accordingly, while the invention is susceptible to various modifications and alternative forms, specific embodiments thereof are shown by way of example in the drawings and will herein be described in detail. It should be understood, however, that there is no intent to limit the invention to the particular forms disclosed, but on the contrary, the invention is to cover all modifications, equivalents, and alternatives falling within the spirit and scope of the invention. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the present invention. As used herein, the term “and/or” includes any and all combinations of one or more of the associated listed items.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (i.e., “between” versus “directly between”, “adjacent” versus “directly adjacent”, etc.).

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

It should also be noted that in some alternative implementations, the functions/acts noted in the blocks may occur out of the order noted in the flowcharts. For example, two blocks shown in succession may in fact be executed substantially concurrently or the blocks may sometimes be executed in the reverse order, depending upon the functionality/acts involved.

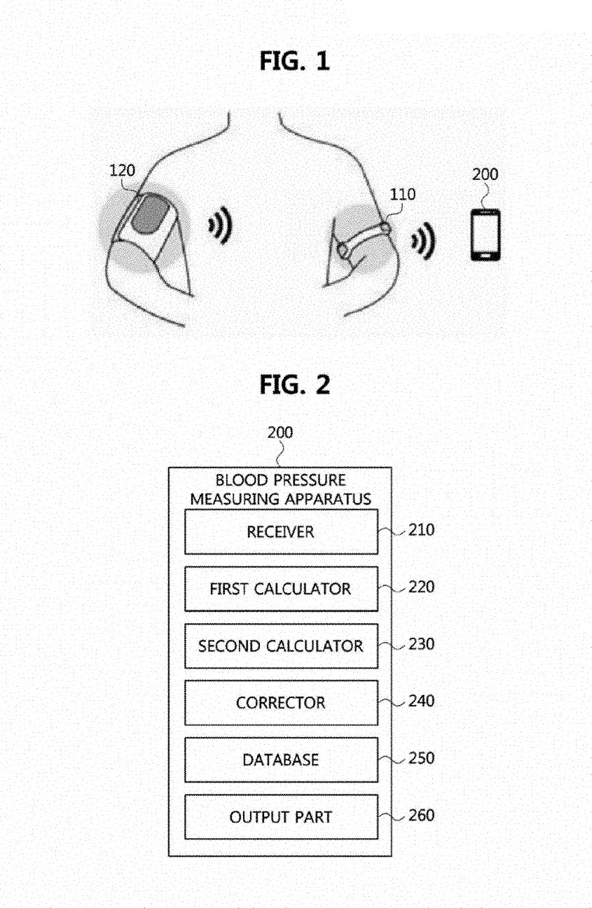

FIG. 1 is a conceptual view illustrating a blood pressure measuring apparatus according to one embodiment of the present invention.

Referring to FIG. 1, a blood pressure measuring apparatus 200 according to one embodiment of the present invention may receive sensing data from a first sensor module 110 and a second sensor module 120 to measure blood pressure of a user. Here, the first sensor module may include an electrocardiogram (ECG) sensor, a photoplethysmogram (PPG) sensor, and a communication module, and the second sensor module may include a pressure sensor and a communication module.

The blood pressure measuring apparatus 200 may be a smart device such as a smart phone including a communication module and a display, or a dedicated device configured to measure blood pressure, but the present invention is not limited thereto. In addition, the blood pressure measuring apparatus 200 may receive the sensing data from the first sensor module 110 and the second sensor module 120, measure the blood pressure of the user on the basis of the sensing data, and provide the measured blood pressure to the user through a display. Specific descriptions about the blood pressure measuring apparatus 200 will be described with reference to FIG. 2.

The first sensor module 110 may include the ECG sensor, the PPG sensor, and the communication module. The first sensor module 110 may continuously obtain ECG information and blood flow rate information of the user using the ECG sensor and the PPG sensor and transmit the sensing data including the ECG information and the blood flow rate information to the blood pressure measuring apparatus 200 using the communication module.

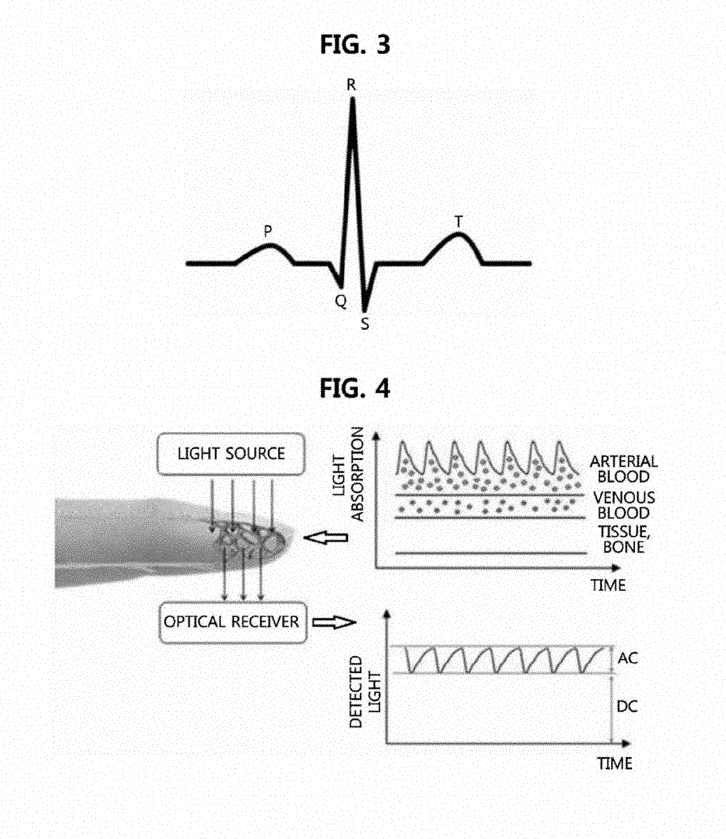

The first sensor module 110 may be manufactured as a band type and may be miniaturized, attached to a body of the user, and operated, or may be manufactured and operated as a wearable type. The first sensor module 110 may obtain the sensing data in a noninvasive, non-pressurization and/or a continuous manner. The first sensor module 110 may transmit the sensing data to the blood pressure measuring apparatus 200 such that the blood pressure measuring apparatus 200 measures the blood pressure of the user, or the first sensor module 110 may also measure the blood pressure of the user using the obtained sensing data and transmit the measured blood pressure information to the blood pressure measuring apparatus 200. A method in which the first sensor module 110 or the blood pressure measuring apparatus 200 measures the blood pressure using the ECG information and the blood flow rate information will be specifically described below with reference to FIGS. 3 to 5.

The second sensor module 120 may include the pressure sensor and the communication module. The second sensor module 120 may obtain pressure information related to blood pressure of the user using the pressure sensor and transmit sensing data including the pressure information to the blood pressure measuring apparatus 200 using the communication module.

The second sensor module 120 may be miniaturized, attached to sportswear, and operated and may obtain the pressure information using a vibrating pressure measuring method. Here, the vibrating pressure measuring method may be a method in which the user applies an air pressure to a cuff which surrounds an upper arm of the user and measures a magnitude of a pressure oscillation generated in the cuff on an arterial vessel through the pressure sensor while the air pressure is slowly lowered. The second sensor module 120 may obtain the sensing data including the pressure information and transmit the sensing data to the blood pressure measuring apparatus 200 such that the blood pressure measuring apparatus 200 may measure the blood pressure of the user, or the second sensor module 120 may also measure the blood pressure of the user using the obtained sensing data and transmit the measured blood pressure information to the blood pressure measuring apparatus 200. A method in which the second sensor module 120 or the blood pressure measuring apparatus 200 measures the blood pressure using the blood pressure information will be specifically described with reference to FIG. 6.

FIG. 2 is a block diagram illustrating the blood pressure measuring apparatus according to one embodiment of the present invention.

Referring to FIG. 2, the blood pressure measuring apparatus 200 according to one embodiment of the present invention may include a receiver 210, a first calculator 220, and a corrector 240, and may further include at least one among a second calculator 230, a database 250, and an output part 260. Here, the components of the blood pressure measuring apparatus 200 are not limited to names thereof, but may be defined by functions thereof. In addition, a plurality of functions may be performed by one component, and one function may also be performed by a plurality of components.

The receiver 210 may include a communication module and receive sensing data from the first sensor module 110 and the second sensor module 120. More specifically, the receiver 210 may receive ECG information and blood flow rate information from the first sensor module 110 and may also receive blood pressure information of the user measured by the first sensor module 110. In addition, the receiver 210 may receive pressure information from the second sensor module 120, or may also receive blood pressure information of the user measured by the second sensor information 120. The communication module of the first sensor module 110, the communication module of the second sensor module 120, and the receiver 210 of the blood pressure measuring apparatus 200 may perform communication using Internet of Things (IoT), but the present invention is not limited thereto, and any general communication method may be used therefor.

The first calculator 220 may calculate blood pressure information of the user on the basis of the ECG information and the blood flow rate information received from the first sensor module 110. In other words, the first calculator 220 may calculate a pulse transit time (PTT) on the basis of the ECG information and the blood flow rate information, calculate a pulse wave velocity (PWV) on the basis of the PTT, and calculate a mean blood pressure (MBP) of the user on the basis of the PWV.

In a case in which the second calculator 230 receives the pressure information from the second sensor module 120, the second calculator 230 may measure blood pressure of the user from the pressure information. In other words, the second calculator 230 may obtain a systolic blood pressure (SBP) value and a diastolic blood pressure (DBP) value of the user from a pressure value detected by the second sensor module 120 using the vibrating pressure measuring method. A method of obtaining the blood pressure value will be specifically described with reference to FIG. 6. However, in a case in which the second sensor module 120 measures the blood pressure of the user using the pressure information and transmits the blood pressure information to the blood pressure measuring apparatus 200, the second calculator 230 may be excluded from the blood pressure measuring apparatus 200.

The corrector 240 may correct the MBP calculated by the first calculator 220 using the blood pressure information obtained from the second sensor module 120 or the blood pressure information calculated by the second calculator 230 to measure the DBP value and the SBP value.

The database 250 may store the blood pressure information including the blood pressure value of the user corrected by the corrector 240. Here, the database 250 may classify and store the blood pressure information according to time, may extract a specific point in time at which abnormal signs and the like are generated, and may also separate the specific point in time from a general point in time to store the specific point in time.

The output part 260 may provide the blood pressure information including the blood pressure value of the user corrected by the corrector 240 to the user in real time and may also provide the blood pressure information of the user according to time or at a specific point in time, which are stored in the database 250, to the user. The output part 260 may provide the above-described information through the display mounted on or connected to the blood pressure measuring apparatus 200 and may also provide the above-described information to an external server spontaneously or according to a demand for requesting an external service.

The blood pressure measuring apparatus 200 according to one embodiment of the present invention may include at least one processor and a memory which stores at least one command configured to perform the above-described operation through the processor. Here, the processor may execute program commands stored in the memory and may be a central processing unit (CPU), a graphics processing unit (GPU), or a specific processor configured to perform the methods according to the present invention. The memory may include a volatile storage media and/or non-volatile storage media such as a read only memory (ROM) and/or a random access memory (RAM).

FIG. 3 is a view illustrating a PQRST wave of the ECG according to one embodiment of the present invention.

The ECG may be a record of an action current measured according to contraction and expansion of myocardium. In other words, the ECG may mean that, in a case in which the action current may flow from the heart to the whole body, the action current may generate a change in potential distribution of the body, and the change may be measured using electrodes attached to the skin of the body.

Referring to FIG. 3, in the ECG, P, Q, R, S, and T waves may be repeatedly recorded. The P wave may express an atrial depolarization period, the Q, R, and S waves may express a ventricular depolarization period, and the T wave may express a ventricular repolarization period.

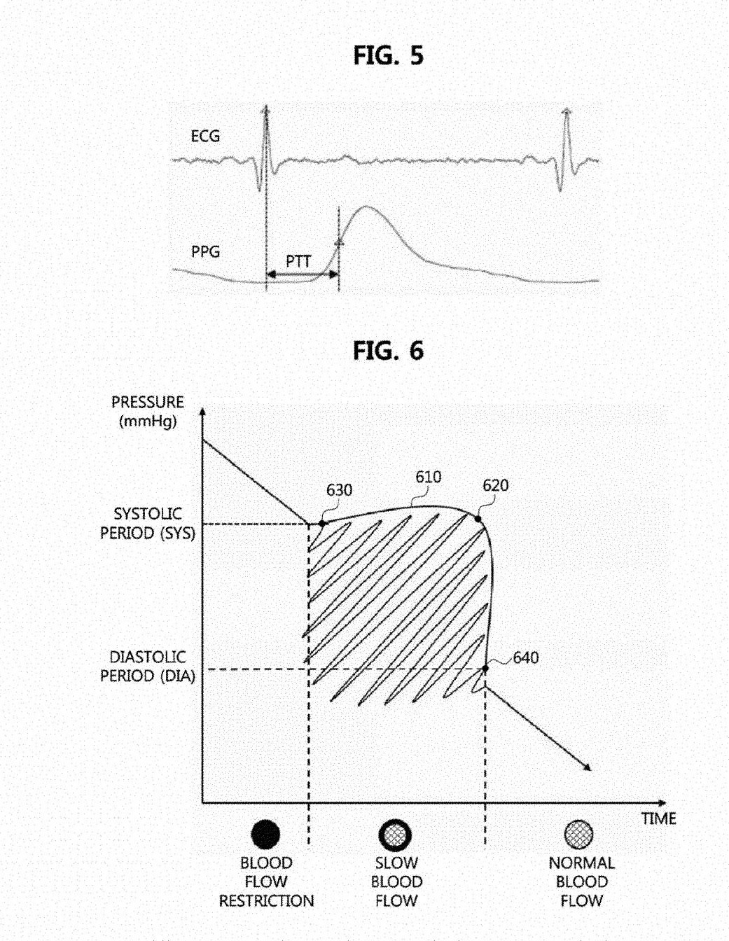

A time at which the R wave is generated in the ECG may be used to calculate a PTT for measuring blood pressure of the user, and a specific method of calculating the PTT will be described with reference to FIG. 5.

FIG. 4 is a conceptual view illustrating the PPG sensor according to one embodiment of the present invention.

The PPG sensor is a kind of an optical sensor that may include a light source configured to emit light and an optical receiver configured to detect light and that may be used for measuring a change in volume of blood flowing through peripheral blood vessels. An operating method of the PPG sensor is as follows. First, the light source may emit light to a specific area of a skin of the user. Some part of the emitted light may be absorbed in a skin region, and the remaining part of the light may penetrate the skin region. The optical receiver may detect the penetrated light and may determine an amount of light absorbed in the skin region on the basis of an amount of emitted light and an amount of light detected by the optical receiver. Here, although the amount of absorbed light may be proportional to skin region, skin tissue, and an amount of blood, a change in blood flow rate may be obtained on the basis of the amount of absorbed light because other factors, except for the change in blood flow rate due to a cardiac impulse, are not changed.

In other words, referring to FIG. 4, although amounts of light absorbed by tissue, bones, and venous blood are constant, since a flow rate of an arterial blood is changed according to the cardiac impulse, the amount of light absorbed by the arterial blood is not constant. Accordingly, in a case in which the optical receiver detects a small amount of light, the optical receiver may determine that a blood flow rate is high in the skin region, and in a case in which the optical receiver detects a large amount of light, the optical receiver may determine that the blood flow rate is low in the skin region.

The blood flow rate information of the user obtained from the PPG sensor may be used to calculate the PTT for measuring the blood pressure of the user, and a specific method of calculating the PTT will be described below with reference to FIG. 5.

FIG. 5 is a view illustrating the PTT according to one embodiment of the present invention.

The PTT may be a time in which a pulse pressure wave or pulse wave is propagated from an aortic valve to a peripheral part of the body and may be measured on the basis of the ECG information and the blood flow rate information obtained by the ECG sensor and the PPG sensor.

Referring to FIG. 5, the PTT may be calculated using a time from (a vertex of) the R wave of the ECG to a specific point in time of the PPG when the ECG information corresponds to the blood flow rate information at the same time.

Here, the specific point in time of the PPG may be a peak value of a primary differentiation, but may also be set by experimentally selecting a most effective value, which is used to estimate blood pressure, among a middle value between a maximum value and a minimum value, a peak value, a peak value of the primary differentiation, and the like. In addition, a noise control filter may be used at a signal processing terminal to select the specific point in time of the PPG.

The blood pressure measuring apparatus 200 according to one embodiment of the present invention may calculate the pulse wave velocity (PWV) on the basis of the above-described PTT in order to measure the blood pressure of the user. The PWV may be a velocity of blood flowing through a blood vessel and may be calculated by Equation 1.

$$PWV = \frac{L}{PTT} \qquad [ Equation 1 ]$$

In Equation 1, PTT may be a pulse transition time, and L may be a distance that blood flows during the PTT.

In addition, the PWV may be expressed as Equation 2 derived from the Moens-Korteweg equation.

$$PWV = \sqrt{\frac{{E}_{in} h}{2 ρr}} \qquad [ Equation 2 ]$$

In Equation 2, Eᵢₙ may be an elastic modulus of an expanded arterial wall, h may be a thickness of an arterial wall, and p may be a density of blood. In addition, r may be a radius of an arterial wall during a diastolic period.

The elastic modulus of the expanded arterial wall and an MBP may have an exponential relation and may be expressed as Equation 3.

Eᵢₙ=E₀e^{γMBP} [Equation 3]

In Equation 3, E₀ and γ may be constants and may be obtained through a cuff-fitting method.

Then, an equation about the MBP may be obtained by combining Equation 1, Equation 2, and Equation 3 and may be expressed as Equation 4.

$$MBP = \frac{1}{γ} ln ( \frac{{L}^{2}}{{PTT}^{2}} \cdot \frac{2 ρr}{{E}_{0} h} ) \qquad [ Equation 4 ]$$

As described above, the blood pressure measuring apparatus 200 according to one embodiment of the present invention may obtain an MBP of the user using the ECG information and the blood flow rate information obtained by the first sensor module 110. Next, a method in which the blood pressure measuring apparatus 200 corrects the MBP on the basis of the pressure information or blood pressure information received from the second sensor module 120 to measure the DBP value and the SBP value will be described.

The equation about the MBP of Equation 4 may be applied to the blood pressure information measured by the second sensor module 120, and in a case in which the equation is applied thereto, the equation may be expressed as Equation 5.

$${MBP}_{0} = \frac{1}{γ} ln ( \frac{{L}^{2}}{{PTT}_{0}^{2}} \cdot \frac{2 ρr}{{E}_{0} h} ) \qquad [ Equation 5 ]$$

In Equation 5, MBP₀ may be an MBP measured using the pressure sensor of the second sensor module 120, and PTT₀ may be a PTT measured in an initial stable state.

A relation between the MBP obtained from the first sensor module 110 and the MBP obtained from the second sensor module 120 may be obtained by combining Equation 4 and Equation 5 and expressed as Equation 6.

$$MBP = {MBP}_{0} + \frac{2}{γ} ln ( \frac{{PTT}_{0}}{PTT} ) \qquad [ Equation 6 ]$$

In addition, the MBP may be expressed on the basis of a DBP and a SBP as in Equation

$$MBP = \frac{1}{3} SBP + \frac{2}{3} DBP \qquad [ Equation 7 ]$$

The DBP and the SBP may be obtained by combining Equation 6 and Equation 7. An equation in which the DBP and the SBP are obtained may be expressed as Equation 8.

$$DBP = \frac{1}{3} {SBP}_{0} + \frac{2}{3} {DBP}_{0} + \frac{2}{γ} ln ( \frac{{PTT}_{0}}{PTT} ) - \frac{( {SBP}_{0} - {DBP}_{0} )}{3} {( \frac{{PTT}_{0}}{PTT} )}^{2} \\ SBP = DBP + ( {SBP}_{0} - {DBP}_{0} ) {( \frac{{PTT}_{0}}{PTT} )}^{2} \qquad [ Equation 8 ]$$

In Equation 8, DBP₀ and SBP₀ may be a DBP value and an SBP value obtained from the second sensor module 120.

FIG. 6 is a graph for describing a vibrating blood pressure measurement method according to one embodiment of the present invention.

In the present invention, the vibrating blood pressure measuring method performed by the pressure sensor of the second sensor module 120 may be a method in which air pressure is applied to the cuff which surrounds the upper arm, and the pressure sensor of the second sensor module 120 measures a magnitude of a pressure oscillation generated in the cuff on the arterial vessel using the pressure sensor to measure blood pressure while the air pressure is slowly lowered. In other words, the vibrating blood pressure measuring method will be specifically described below.

First, air may be injected into the cuff until the cuff has a pressure which exceeds a systolic arterial pressure to restrict flow of blood. Then, when the air is slowly discharged and the pressure of the cuff starts to decrease under the systolic arterial pressure and the blood starts to flow again, periodic arterial contraction and expansion are measured from the starting time using the pressure sensor until the pressure of the cuff decreases under the diastolic arterial pressure.

Referring to FIG. 6, it may be seen that a wave is generated according to the periodic contraction and expansion of the artery while the pressure gradually decreases as the air is slowly discharged. Here, the second sensor module 120 may record an arc 610 of the wave and a most high wave 620 according to the periodic contraction and expansion of the artery. In addition, a point 630 from which the wave starts may be measured as a systolic arterial pressure, and a point 640 at which the wave ends may be measured as a diastolic arterial pressure. The wave will be described below with flow of the blood. It may be seen that the blood may not flow because the flow of the blood is restricted before the wave is generated, the blood may start to gradually flow because the air pressure gradually decreases in a section in which the wave is generated, and the blood flows normally after the wave is generated.

The second sensor module 120 may measure the pressure through the above-described vibrating pressure measuring method and transmit the measured pressure to the blood pressure measuring apparatus 200, or the second sensor module 120 may obtain blood pressure information of the user on the basis of the measured pressure and transmit the blood pressure information to the blood pressure measuring apparatus 200. Here, the blood pressure information may include at least one among an MBP value, an SBP value, and a DBP value.

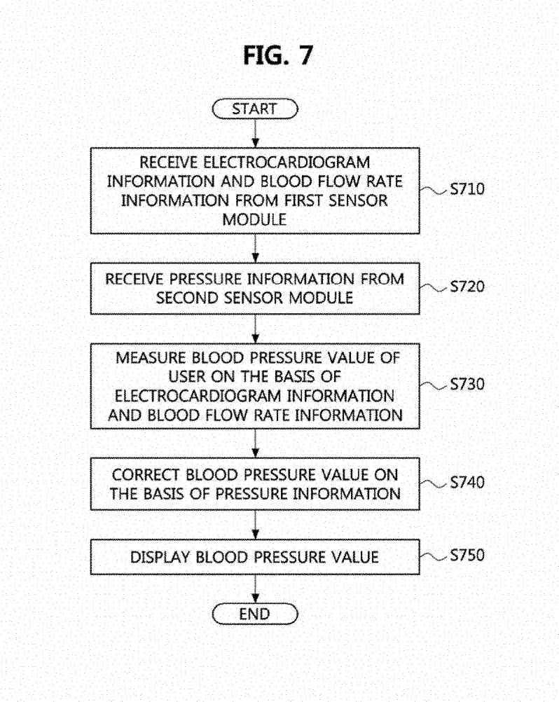

Here, the second sensor module 120 may measure the pressure one time at each specific point in time or specific period, for example, two to three times per day, and transmit sensing data to the blood pressure measuring apparatus 200. In other words, continuous blood pressure measurements performed by the first sensor module 110 may be main measurements, and blood pressure measurements performed by the second sensor module 120 may be sub-measurements for correcting a blood pressure value measured by the first sensor module 110. FIG. 7 is a flowchart illustrating a method of measuring blood pressure according to one embodiment of the present invention.

Referring to FIG. 7, the method of measuring the blood pressure performed by the blood pressure measuring apparatus 200 according to one embodiment of the present invention may include receiving ECG information and blood flow rate information from the first sensor module 110 (S710) and receiving pressure information from the second sensor module (S720). However, in a case in which the second sensor module 120 obtains blood pressure information of a user from the pressure information and transmits the blood pressure information to the blood pressure measuring apparatus 200, the blood pressure measuring apparatus 200 may receive the blood pressure information.

The blood pressure measuring apparatus 200 may measure the blood pressure value of the user on the basis of the received ECG information and the received blood flow rate information (S720) and may correct the measured blood pressure value on the basis of the received pressure information (S730). Since the present invention measures the blood pressure value and also corrects the blood pressure value, the present invention may relatively accurately measure the blood pressure value. However, in a case in which the second sensor module 120 transmits the blood pressure information, the blood pressure measuring apparatus 200 may correct the measured blood pressure value on the basis of the blood pressure information. Here, the blood pressure value may include at least one among an MBP, a DBP, and an SBP.

Then, the blood pressure measuring apparatus 200 according to one embodiment of the present invention may display the measured and corrected blood pressure value and provide the blood pressure value to the user (S750) and may also transmit the blood pressure value to an external server spontaneously or according to a demand for an external service.

According to the example embodiments of the present invention, since blood pressure can be continuously measured for 24 hours, abnormal signs of the blood pressure can be easily checked.

A non-dipper type, in which blood pressure does not lower at night, can be checked.

A phenomenon of white-coat hypertension, wherein blood pressure is measured to be high only in a doctor's office, or a masked hypertension phenomenon, wherein blood pressure is measured to be normal only in a doctor's office, can be prevented, and blood pressure can be accurately measured.

Since a variability depending on a measurement environment, a measurement site, and a clinical situation can be considered when blood pressure is measured, blood pressure can be accurately measured.

While the example embodiments of the present invention and their advantages have been described in detail, it should be understood that various changes, substitutions and alterations may be made herein without departing from the scope of the invention.

What is claimed is:

1. A method of measuring blood pressure comprising:
receiving electrocardiogram information and blood flow rate information from a first sensor module;
calculating a pulse transit time on the basis of the electrocardiogram information and the blood flow rate information;
calculating a mean blood pressure value of a user on the basis of the pulse transit time;
receiving a reference data from a second sensor module; and
calculating a diastolic blood pressure value and a systolic blood pressure value of a user on the basis of the mean blood pressure value and the reference data.

2. The method of claim 1, wherein the calculating of the pulse transit time on the basis of the electrocardiogram information and the blood flow rate information received from the first sensor module includes:
synchronizing the electrocardiogram information and the blood flow rate information; and
calculating a pulse transit time which is a time period from a point in time at which an R wave of the electrocardiogram information starts to a specific point in time of the blood flow rate information.

3. The method of claim 2, wherein the specific point in time of the blood flow rate information is a point in time at which a primary differential value of a function in which the blood flow rate information is expressed according to time is maximized.

4. The method of claim 1, wherein the calculating of the mean blood pressure value of the user on the basis of the pulse transit time includes:
calculating a pulse wave velocity on the basis of the pulse transit time; and
calculating the mean blood pressure value on the basis of the pulse transit time and the pulse wave velocity.

5. The method of claim 1, wherein the first sensor module includes:
an electrocardiogram sensor; and
a photoplethysmogram sensor.

6. The method of claim 1, wherein the second sensor module includes a pressure sensor.

7. The method of claim 6, wherein the reference data includes a reference mean blood pressure value, a reference diastolic blood pressure value, and a reference systolic blood pressure value which are measured by the second sensor module using the pressure sensor through a vibrating pressure measuring method.

8. The method of claim 1, further comprising providing at least one among the mean blood pressure value, the diastolic blood pressure value, and the systolic blood pressure value to the user or an external server.

9. A blood pressure measuring apparatus comprising:
a receiver configured to receive electrocardiogram information and blood flow rate information from a first sensor module and receive reference data from a second sensor module;
a first calculator configured to calculate a pulse transit time on the basis of the electrocardiogram information and the blood flow rate information and calculate a mean blood pressure value of a user on the basis of the pulse transit time; and
a corrector configured to calculate a diastolic blood pressure value and a systolic blood pressure value of a user on the basis of the mean blood pressure value and the reference data.

10. The blood pressure measuring apparatus of claim 9, wherein the first calculator:
synchronizes the electrocardiogram information and the blood flow rate information; and
calculates a pulse transit time which is a time period from a point in time at which an R wave of the electrocardiogram information starts to a specific point in time of the blood flow rate information.

11. The blood pressure measuring apparatus of claim 10, wherein the specific point in time of the blood flow rate information is a point in time at which a primary differential value of a function in which the blood flow rate information is expressed according to time is maximized.

12. The blood pressure measuring apparatus of claim 10, wherein the first calculator calculates:
a pulse wave velocity on the basis of the pulse transit time; and
the mean blood pressure value on the basis of the pulse transit time and the pulse wave velocity.

13. The blood pressure measuring apparatus of claim 9, wherein the first sensor module includes:
an electrocardiogram sensor; and
a photoplethysmogram sensor.

14. The blood pressure measuring apparatus of claim 9, wherein the second sensor module includes a pressure sensor.

15. The blood pressure measuring apparatus of claim 14, wherein the reference data includes a reference mean blood pressure value, a reference diastolic blood pressure value, and a reference systolic blood pressure value which are measured by the second sensor module using the pressure sensor through a vibrating pressure measuring method.

16. The blood pressure measuring apparatus of claim 9, further comprising an output part configured to provide at least one among the mean blood pressure value, the diastolic blood pressure value, and the systolic blood pressure value to the user or an external server.
